(19) United States
(12) Patent Application Publication
(10) Pub. No.: US 2021/0031039 A1
(43) Pub. Date: Feb. 4, 2021

(54) COMPARISON TECHNIQUES FOR PROSTHESIS FITTING

(71) Applicant: COCHLEAR LIMITED, New South Wales (AU)

(72) Inventor: Sean LINEAWEAVER, Macquarie University (AU)

(21) Appl. No.: 16/964,553

(22) PCT Filed: Jan. 24, 2019
(86) PCT No.: PCT/IB2019/050601

§ 371 Date: Jul. 23, 2020

Related U.S. Application Data
(60) Provisional application No. 62/621,256, filed on Jan. 24, 2018.

(51) Int. Cl. A61N 1/36; H04R 25/00

(52) U.S. Cl. CPC A61N 1/36039; A61N 1/36046; H04R 25/70

(57) ABSTRACT

A method including obtaining data relating to a parameter having a first variable and a parameter having a second variable different from the first variable, and developing fitting data for a sense prosthesis for an individual based on the obtained data utilizing a tabu algorithm. In an exemplary embodiment, the sense prosthesis includes a cochlear implant and the obtained data is data relating to electric hearing and data relating to acoustic hearing.

### CROSS-REFERENCE TO RELATED APPLICATIONS

This application claims priority to U.S. Provisional Application No. 62/621,256, entitled COMPARISON TECHNIQUES FOR PROSTHESIS FITTING, filed on Jan. 24, 2018, naming Sean LINEAWEAVER of Gig Harbor, Wash. as an inventor, the entire contents of that application being incorporated herein by reference in its entirety.

### BACKGROUND

Hearing loss, which may be due to many different causes, is generally of two types: conductive and sensorineural. Sensorineural hearing loss is due to the absence or destruction of the hair cells in the cochlea that transduce sound signals into nerve impulses. Various hearing prostheses are commercially available to provide individuals suffering from sensorineural hearing loss with the ability to perceive sound. One example of a hearing prosthesis is a cochlear implant.

Conductive hearing loss occurs when the normal mechanical pathways that provide sound to hair cells in the cochlea are impeded, for example, by damage to the ossicular chain or the ear canal. Individuals suffering from conductive hearing loss may retain some form of residual hearing because the hair cells in the cochlea may remain undamaged.

Individuals suffering from hearing loss typically receive an acoustic hearing aid. Conventional hearing aids rely on principles of air conduction to transmit acoustic signals to the cochlea. In particular, a hearing aid typically uses an arrangement positioned in the recipient's ear canal or on the outer ear to amplify a sound received by the outer ear of the recipient. This amplified sound reaches the cochlea causing motion of the perilymph and stimulation of the auditory nerve. Cases of conductive hearing loss typically are treated by means of bone conduction hearing aids. In contrast to conventional hearing aids, these devices use a mechanical actuator that is coupled to the skull bone to apply the amplified sound.

In contrast to hearing aids, which rely primarily on the principles of air conduction, certain types of hearing prostheses commonly referred to as cochlear implants convert a received sound into electrical stimulation. The electrical stimulation is applied to the cochlea, which results in the perception of the received sound.

Many devices, such as medical devices that interface with a recipient, have structural and/or functional features where there is utilitarian value in adjusting such features for an individual recipient. The process by which a device that interfaces with or otherwise is used by the recipient is tailored or customized or otherwise adjusted for the specific needs or specific wants or specific characteristics of the recipient is commonly referred to as fitting. One type of medical device where there is utilitarian value in fitting such to an individual recipient is the above-noted cochlear implant. That said, other types of medical devices, such as other types of hearing prostheses and retinal prostheses, exist where there is utilitarian value in fitting such to the recipient.

### SUMMARY

In accordance with an exemplary embodiment, there is a method, comprising obtaining data relating to a parameter having a first variable and a parameter having a second variable different from the first variable and developing fitting data for a sense prosthesis for an individual based on the obtained data utilizing a tabu algorithm.

In accordance with another exemplary embodiment, there is a method, comprising setting a cochlear implant to operate based on data based on a first number of comparisons between respective data sets respectively including combined data for electric stimulation to evoke a hearing percept and data for acoustic stimulation to evoke a hearing percept, wherein the first number of comparisons is less than a total number of possible comparisons resulting from all possible permutations of the controlled variables that make up the respective data sets.

In accordance with another exemplary embodiment, there is a non-transitory computer readable medium having recorded thereon, a computer program for executing a method, the program including code for executing a tabu algorithm to develop respective test data to fit a sense prosthesis to a recipient, wherein the respective test data includes respective sensory percepts to be evoked using the prosthesis, wherein the respective test data is at least two dimensional data.

In accordance with another exemplary embodiment, there is a fitting system, comprising a first sub-system configured to cause respective stimulus groups to be applied by a sensory prosthesis to a recipient of the sensory prosthesis and a second sub-system configured to automatically determine the respective stimulus groups to be applied from a pool of potential respective stimulus groups that is larger than what the first sub-system will cause to be applied.

### BRIEF DESCRIPTION OF THE DRAWINGS

Embodiments are described below with reference to the attached drawings, in which:

FIG. 1A is a perspective view of an exemplary multimodal hearing prosthesis according to an exemplary embodiment;

FIG. 1B is another view of the exemplary multimodal hearing prosthesis presented in FIG. 1A;

FIG. 1C provides additional details of the exemplary multimodal hearing prosthesis of FIG. 1B;

FIG. 2 presents an exemplary data chart for a person with residual hearing;

FIG. 3 presents an exemplary audiogram for a person with residual hearing;

FIG. 4 presents a flowchart for a method;

FIGS. 5-8 present conceptual charts;

FIGS. 9 and 10 present respective graphics illustrating a function of a tabu algorithm;

FIG. 11 presents an exemplary flowchart for a method; and

FIG. 12 presents an exemplary flowchart for a method.

### DETAILED DESCRIPTION

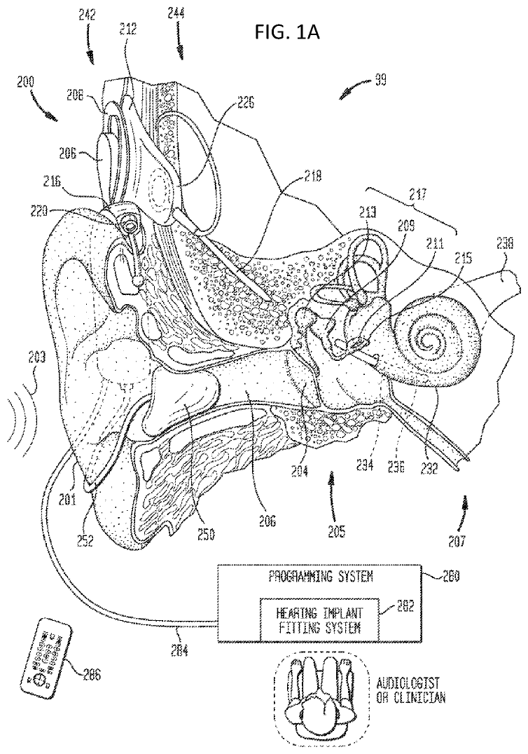

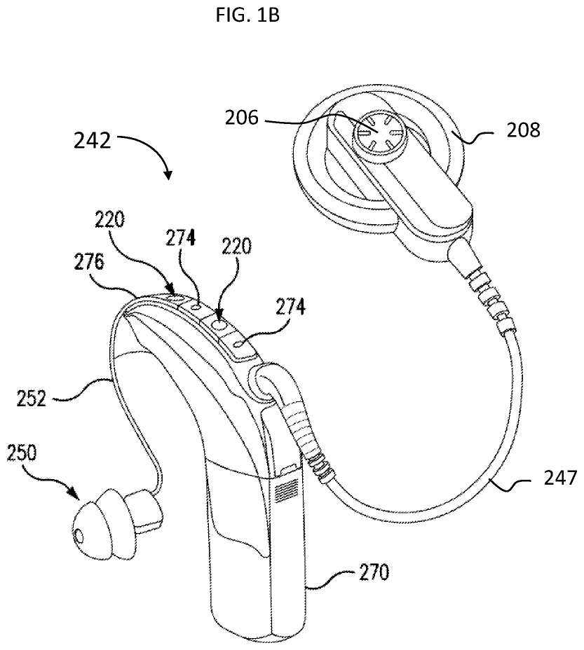

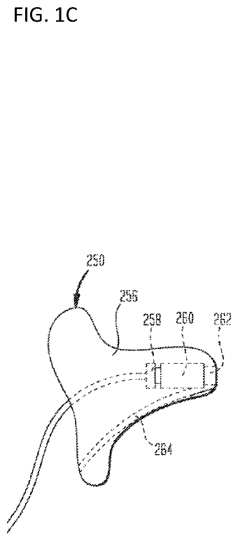

FIG. 1A is a perspective view of an implanted multimodal system 200 that includes a cochlear implant system implanted in a recipient, to which some embodiments detailed herein and/or variations thereof are applicable. The cochlear implant system can include external components in some embodiments, and implanted component (internal/implantable component), as will be detailed below. Additionally, it is noted that the teachings detailed herein are also applicable to other types of hearing prostheses, such as by way of example only and not by way of limitation, bone conduction devices (percutaneous, active transcutaneous, and/or passive transcutaneous), direct acoustic cochlear stimulators, middle ear implants, and conventional hearing aids, etc. (and a multimodal system can include two or more of the aforementioned combinations, including a cochlear implant combined with another device) at least with respect to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing. Accordingly, any disclosure herein with regard to one of these types of hearing prostheses corresponds to a disclosure of another of these types of hearing prostheses or any medical device for that matter, unless otherwise specified, or unless the disclosure thereof is incompatible with a given device based on the current state of technology. Thus, the teachings detailed herein are applicable, in at least some embodiments, to partially implantable and/or totally implantable medical devices that provide a wide range of therapeutic benefits to recipients, patients, or other users, including hearing implants having an implanted microphone, auditory brain stimulators, pacemakers, visual prostheses (e.g., bionic eyes), sensors, drug delivery systems, defibrillators, functional electrical stimulation devices, catheters, etc.

In view of the above, it is to be understood that at least some embodiments detailed herein and/or variations thereof are directed towards a body-worn sensory supplement medical device (e.g., the hearing prosthesis of FIG. 1A, which supplements the hearing sense, even in instances when there are no natural hearing capabilities, for example, due to degeneration of previous natural hearing capability or to the lack of any natural hearing capability, for example, from birth). It is noted that at least some exemplary embodiments of some sensory supplement medical devices are directed towards devices such as conventional hearing aids, which supplement the hearing sense in instances where some natural hearing capabilities have been retained, and visual prostheses (both those that are applicable to recipients having some natural vision capabilities and to recipients having no natural vision capabilities). Accordingly, the teachings detailed herein are applicable to any type of sensory supplement medical device to which the teachings detailed herein are enabled for use therein in a utilitarian manner. In this regard, the phrase sensory supplement medical device refers to any device that functions to provide sensation to a recipient irrespective of whether the applicable natural sense is only partially impaired or completely impaired, or indeed never existed.

Again with respect to FIG. 1A, FIG. 1A is a perspective view of an exemplary multimodal prosthesis in which the present invention may be implemented. The ear 99 includes outer ear 201, middle ear 205, and inner ear 207 are described next below, followed by a description of an implanted multimodal system 200. Multimodal system 200 provides multiple types of stimulation, i.e., acoustic, electrical, and/or mechanical. These different stimulation modes may be applied ipsilaterally or contralaterally. In the embodiment shown in FIG. 1A, multimodal implant 200 provides acoustic and electrical stimulation, although other combinations of modes can be implemented in some embodiments. By way of example and not by way of limitation, a middle-ear implant can be utilized in combination with the cochlear implant, a bone conduction device can be utilized in combination with the cochlear implant, etc.

In a person with normal hearing or a recipient with residual hearing, an acoustic pressure or sound wave 203 is collected by outer ear 201 (that is, the auricle) and channeled into and through ear canal 206. Disposed across the distal end of ear canal 206 is a tympanic membrane 204 which vibrates in response to acoustic wave 203. This vibration is coupled to oval window, fenestra ovalis 215 through three bones of middle ear 205, collectively referred to as the ossicles 217 and comprising the malleus 213, the incus 209, and the stapes 211. Bones 213, 209, and 211 of middle ear 205 serve to filter and transfer acoustic wave 203, causing oval window 215 to articulate, or vibrate. Such vibration sets up waves of fluid motion within cochlea 232. Such fluid motion, in turn, activates tiny hair cells (not shown) that line the inside of cochlea 232. Activation of the hair cells causes appropriate nerve impulses to be transferred through the spiral ganglion cells (not shown) and auditory nerve 238 to the brain (not shown), where such pulses are perceived as sound.

In individuals with a hearing deficiency who may have some residual hearing, an implant or hearing instrument may improve that individual's ability to perceive sound. Multimodal prosthesis 200 may comprise external component assembly 242 which is directly or indirectly attached to the body of the recipient, and an internal component assembly 244 which is temporarily or permanently implanted in the recipient. External component assembly is also shown in FIG. 1B. In embodiments of the present invention, components in the external assembly 242 may be included as part of the implanted assembly 244, and vice versa. Also, embodiments of the present invention may be used with implanted multimodal system 200 which are fully implanted.

External assembly 242 typically comprises a sound transducer 220 for detecting sound, and for generating an electrical audio signal, typically an analog audio signal. In this illustrative embodiment, sound transducer 220 is a microphone. In alternative embodiments, sound transducer 220 can be any device now or later developed that can detect sound and generate electrical signals representative of such sound.

External assembly 242 also comprises a signal processing unit, a power source (not shown), and an external transmitter unit. External transmitter unit 206 comprises an external coil 208 and, preferably, a magnet (not shown) secured directly or indirectly to the external coil 208. The signal processing unit processes the output of microphone 220 that is positioned, in the depicted embodiment, by outer ear 201 of the recipient. The signal processing unit generates coded signals, referred to herein as a stimulation data signals, which are provided to external transmitter unit 206 via a cable 247 and to the receiver in the ear 250 via cable 252. FIG. 1C provides additional details of an exemplary receiver 250. The overall component containing the signal processing unit is, in this illustration, constructed and arranged so that it can fit behind outer ear 201 in a BTE (behind-the-ear) configuration, but may also be worn on different parts of the recipient's body or clothing.

In some embodiments, the signal processor may produce electrical stimulations alone, without generation of any acoustic stimulation beyond those that naturally enter the ear. While in still further embodiments, two signal processors may be used. One signal processor is used for generating electrical stimulations in conjunction with a second speech processor used for producing acoustic stimulations.

As shown in FIGS. 1B and 1C, a receiver in the ear 250 is connected to the signal processor through cable 252. Receiver in the ear 250 includes a housing 256, which may be a molding shaped to the recipient. Inside the receiver in the ear 250 there is provided a capacitor 258, receiver 260 and protector 262. Also, there may be a vent shaft 264 (in some embodiments, this vent shaft is not included). Receiver in the ear may be an in-the-ear (ITE) or completely-in-canal (CIC) configuration.

Also, FIG. 1B shows a removable battery 270 directly attached to the body/spine of the BTE device. As seen, the BTE device in some embodiments control buttons 274. In addition, the BTE may house a power source (not shown), e.g., zinc-air batteries. The BTE device may have an indicator light 276 on the earhook to indicate operational status of the signal processor. Examples of status indications include a flicker when receiving incoming sounds, low rate flashing when power source is low or high rate flashing for other problems.

Returning to FIG. 1A, internal components 244 comprise an internal receiver unit 212, a stimulator unit 226 and an electrode assembly 218. Internal receiver unit 212 comprises an internal transcutaneous transfer coil (not shown), and preferably, a magnet (also not shown) fixed relative to the internal coil. Internal receiver unit 212 and stimulator unit 226 are hermetically sealed within a biocompatible housing. The internal coil receives power and data from external coil 208, as noted above. A cable or lead of electrode assembly 218 extends from stimulator unit 226 to cochlea 232 and terminates in an array 234 of electrodes 236. Electrical signals generated by stimulator unit 226 are applied by electrodes 236 to cochlea 232, thereby stimulating the auditory nerve 238.

In one embodiment, external coil 208 transmits electrical signals to the internal coil via a radio frequency (RF) link. The internal coil is typically a wire antenna coil comprised of at least one and preferably multiple turns of electrically insulated single-strand or multi-strand platinum or gold wire. The electrical insulation of the internal coil is provided by a flexible silicone molding (not shown). In use, internal receiver unit 212 may be positioned in a recess of the temporal bone adjacent to outer ear 201 of the recipient.

As shown in FIG. 1A, multimodal system 200 is further configured to interoperate with a user interface 280 and an external processor 282 such as a personal computer, workstation, or the like, implementing, for example, a hearing implant fitting system. Although a cable 284 is shown in FIG. 1A between implant 200 and interface 280, a wireless RF communication may also be used along with remote 286.

While FIG. 1A shows a multimodal implant in the ipsilateral ear, in other embodiments of the present invention the multimodal implant may provide stimulation to both ears. For example, a signal processor may provide electrical stimulation to one ear and provide acoustical stimulation in the other ear.

Using an exemplary multimodal device shown in FIGS. 1A and 1B, the prescription process that embodiments of the present invention may use is described in the following systems and methods.

In at least some exemplary embodiments, there is utilitarian value with respect to determining what frequency bands the multimodal prosthesis 200, or single mode prosthesis, will allocate towards electric hearing (e.g., hearing based on the utilization of the electrode assembly 218) and acoustic hearing (e.g., hearing that is prompted by the in the ear device 250 in general, and the projector 262 in particular and/or hearing that will be left to natural means (e.g., no amplification)). It is noted that by allocating frequency bands to acoustic hearing, this can include leaving those frequency bands to natural hearing (such as in the single mode prosthesis, where, for example, the prosthesis is a cochlear implant). That is, the multimodal prosthesis 200 (or single mode prosthesis—hereinafter, reference to a multimodal prosthesis also constitutes disclosure of a single mode prosthesis, such as a cochlear implant) can be such that frequency bands allocated to acoustic hearing simply result in no action by the prosthesis 200 at all. That said, in at least some exemplary embodiments, such as those that utilize the ITE device 250, those frequency bands for acoustic hearing will be provided to the ITE device 250 so that the projector 262 can output an acoustic signal in an amplified manner to evoke a hearing percept akin to that which corresponds to the utilization of a conventional hearing aid, at least for those channels/frequencies.

It is noted that in some embodiments, there is overlap between the electric hearing frequencies and the acoustic hearing frequencies.

The utilitarian value associated with determining what frequency bands the multimodal prosthesis 200 will allocate towards electric hearing and acoustic hearing can result in the maximizing of hearing. Hereinafter, the “bifurcation” between acoustic hearing and electric hearing is sometimes referred to as the acoustic-to-electric cross-over frequency or the acoustic-to-electric boundary (sometimes, AE cross-over or AE boundary, for short—it is noted that sometimes, this is also referred to as the electro-acoustic boundary, or EA boundary for short—both mean the same thing). It is noted that the term “bifurcation” is utilized loosely in that in some instances, the bifurcation can result in the overlap between the two types of hearing, as will be described in greater detail below. In this regard, there is utilitarian value associated with determining an overlap of the electrical hearing with the acoustic hearing. For example, in a given scenario where the AE boundary is set at 1,000 Hz (herein, the AE boundary is such that the acoustic stimulation is lower than the electrical stimulation, frequency wise) there can be utilitarian value with respect to having 1, 2, 3, 4, 5, 6, 7, 8, 9, 10 or more stimulating electrodes in the acoustic areas, although in some other exemplary embodiments, there can be utilitarian value with respect to having no overlap.

In general terms, in a given exemplary scenario of use, for a newly implanted cochlear implant recipient with residual hearing in the implanted ear (or another ear—more on this below), there can be utilitarian value with respect to determining the acoustic-to-electric cross-over frequency (AE boundary). Again, it is noted that while the teachings detailed herein are described in terms of the multimodal prosthesis 200, that includes the ITE 250 with the projector 262, in some alternate embodiments, the teachings detailed herein are applicable to a unimodal (single mode) prosthesis corresponding to a cochlear implant without an acoustic hearing aid (receiver 250 having the projector 262). By way of example only and not by way of limitation, in an exemplary embodiment, the teachings detailed herein can be utilized in a single mode cochlear implant prostheses were that is the only prostheses that the recipient has and/or where the recipient has no residual hearing. For example, the teachings detailed herein can be utilized to compare whether it is better to utilize all electrodes of an electrode array (e.g., all 22 electrodes of a 22 electrode array), or at least most of them (e.g., 21 or 20 or 19—there are often a few channels that do not work for whatever reason), verses utilizing subsets of electrodes of the electrode array (e.g., 10 electrodes of the 22 electrodes, 8 of the 22, 12 of the 22, etc.).

In some exemplary embodiments where the recipient has residual hearing, the prosthesis, whether such is a multimodal prosthesis 200 or unimodal prosthesis in the form of a cochlear implant, the prosthesis is “fitted” to the recipient. The details of such fitting entail activating the prosthesis in general, and the electrode array/electrode assembly 218 of the cochlear implant in particular, while implanted in the recipient, to evoke a hearing percept, and adjust settings of the prosthesis based on the particular recipient's physiology/reactions to the stimulus from the prosthesis. In at least some exemplary embodiments, this entails setting so-called threshold and comfort levels. In at least some exemplary embodiments, this entails tonotopically mapping the various channels of the cochlear implant. The teachings detailed herein are applicable to pre-fitting actions associated with the prosthesis. Indeed, in an exemplary embodiment, the teachings are directed towards developing a prescription for a given recipient, which prescription will be used to fit the recipient (the prescription might be contemporaneous with the fitting). Herein, any reference to a development of a prescription also corresponds to a disclosure of developing fitting data for the prostheses, and vice versa, as the two can be utilized simultaneously in at least some exemplary embodiments.

Some exemplary embodiments are directed towards developing a prescription (and/or fitting settings) that balances acoustic hearing with electric hearing, based on the subjective needs of the recipient while also identifying the potential for an overlap between the electric hearing in the acoustic hearing, at least with respect to the AE boundary.

It is noted that the existence of the ability to hear utilizing natural hearing/to hear utilizing acoustic stimulus can in some instances be a result of the implantation procedure/location of the cochlear implant electrode array. By way of example only and not by way of limitation, in some instances, a so called short electrode array is utilized, which electrode array does not extend into the cochlea as far as a full-length electrode array. By way of example only and not by way of limitation, such an electrode array can be configured to stimulate at the high frequency locations within the cochlea, and that is all, owing to the fact that the electrodes, and thus the electrode array, does not extend the typical full distance into the cochlea. By way of example only and not by way of limitation, a so called short electrode array could have 4, 5, 6, 7, 8, 9, 10, 11, 12, 13, 14, or 15 electrodes, as opposed to, for example, the “normal” 22 electrodes of, for example, a full array made and produced by Cochlear LTD, such as the array for the Nucleus 5® cochlear implant. The aforementioned electrodes of the short electrode array would be positioned such that those respective electrodes on the topically mapped as would be the case with respect to similar electrodes of the Nucleus 5® cochlear implant beginning at the basal location, and thus, the electrodes for high-frequency stimulation would be present in both types of electrodes—it is the electrodes for the low frequency stimulation that may not be present.

Accordingly, in the embodiments of the short electrode array, because the array does not extend into the medium and low frequency locations in the cochlear, or because the array does not extend into the low frequency locations of the cochlea, the ability to hear naturally/to hear by acoustic stimulation at those frequencies is more likely to remain than that which would be the case if a full-length electrode array extending to those locations was implanted into the recipient.

The above said, in some embodiments, the position of the electrode array is such that the electrode array does not contact the modiolus wall of the cochlea (e.g., by way of example only and not by way of limitation, a so-called lateral wall cochlea array insertion), and thus even though the electrode array extends into the medium and low frequency regions or low frequency regions, the ability to hear naturally/to hear by acoustic stimulation is maintained or otherwise is more likely to be maintained than that which would be the case if the electrode array having such full-length was inserted in a manner that the electrode array directly contacted the modiolus wall.

It is also noted that even with respect to “no overlap,” this phrase is utilized with a qualification that there might be some residual hearing in the electric stimulation region, but this residual hearing is below a threshold that is established, sometimes arbitrarily. Accordingly, let us now discuss the beginning and background concepts.

Multi-modal devices that utilize cochlear implants can be implemented for use by people with some high-frequency hearing loss (in some instances, clinically severe loss) but who still have the ability to hear lower frequencies, in some instances, with minimal assistance, and in other instances, with assistance. In this regard, an exemplary embodiment includes a cochlear implant that includes an integrated sound processor (whether in the implanted part or in the external part) that presents sound information via a combined acoustic and electric signal to recipients who have functional low frequency hearing. This can provide recipients with acoustically amplified sound across the frequency range 125->6000 Hz and electrical stimulation at higher frequencies via electrical stimulation.

Electric hearing can restore access to audibility to the high frequencies that are utilitarian for speech understanding. The acoustic signal provided by such an implant or simply the acoustic signal that results from the fact that the implant has been implanted in such a way as to retain a residual hearing at summer all frequencies can provide low frequency temporal fine structure information that currently is not conveyed in the electrical signal. This increased low frequency spectral resolution is utilitarian for musical and/or voice pitch perception. The acoustic signal can sometimes better represent pitch and/or fundamental frequency (F0) and frequency selectivity (F1 [270-˜1000 Hz] vowel cues and low frequency consonant cues such as those for voicing and manner) which together can, in some embodiments, enable a listener to take advantage of pitch differences between speakers and to segregate speech targets from noise. These low frequency cues can contribute to improved speech understanding (relative to the absence of such), such as in scenarios where there is background noise. In addition, low frequency acoustic information can give a more natural sound quality compared to electric hearing alone.

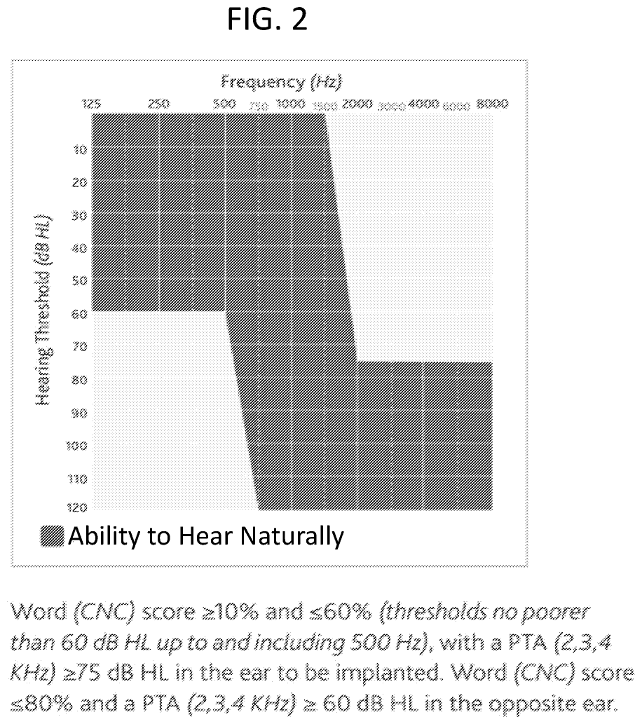

FIG. 2 presents an exemplary chart that shows hearing threshold versus frequency. In an exemplary embodiment, a recipient having the ability to hear according to the chart of FIG. 2 could be a candidate for a multimodal hearing device (again, which as used herein, also corresponds to a disclosure of a single mode hearing device that permits residual hearing).

The data associated with FIG. 2 can be developed, in some exemplary embodiments, by executing a so-called fitting routine of a recipient having the given prostheses. In an exemplary embodiment, fitting the prosthesis includes meeting the “requirements” for the acoustic frequencies. As such, in an exemplary embodiment, in some form or another, and acoustic audiogram is obtained. In some exemplary embodiments, this is executed in a manner the same as or otherwise consistent with fitting a conventional acoustic hearing aid. By way of example only and not by way of limitation, Real Ear™ (or alternately Sound Field™) measurements are used to determine the acoustic hearing loss at different frequencies.

An exemplary embodiment is such that a rigorous audiogram is used, which measures hearing loss at the following frequencies (by way of example only): 250 Hz, 500 Hz, 750 Hz, 1000 Hz, 1500 Hz, 2000 Hz, 4000 Hz, and 8000 Hz. it is noted that in some exemplary embodiments, the audiogram can measure hearing loss at other frequencies and/or at more frequencies. Any measurements that can have utilitarian value with respect to implementing the teachings detailed herein can be utilized at least some exemplary embodiments.

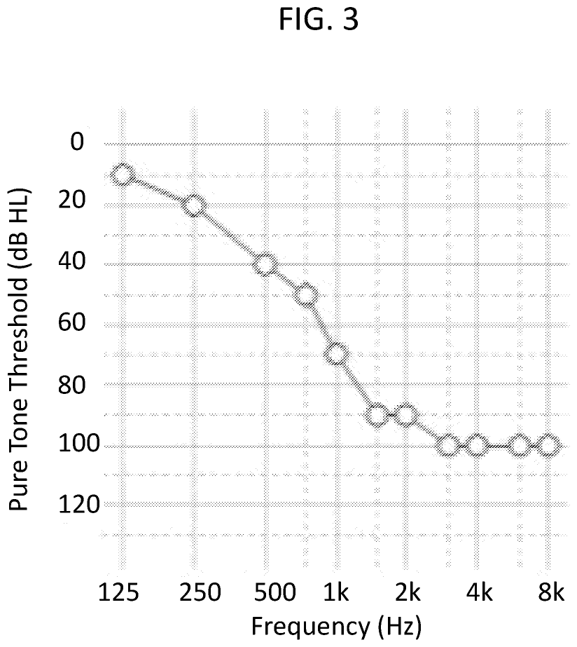

For multi-modal fitting, such can be executed by measuring hearing deficit below 90 dB HL out to 2200 Hz. In an exemplary embodiment, the AE boundary is designated at the frequency where the acoustic audiogram threshold level drops below 70 dB HL. FIG. 3 provides an exemplary audiogram of such a scenario.

In the example audiogram presented in FIG. 3, the AE boundary occurs at 1000 Hz (because the boundary for acoustic hearing has been determined to be those frequencies where the acoustic audiogram has not dropped below 70 dB HL—thus, 1000 Hz is the first data point where the audiogram indicates a threshold level that is not below 70 dB—in reality, it is possible that the recipient's threshold level hearing is below 70 dB for frequencies at, for example 990 Hz, 980 Hz, etc., but those frequencies were not measured—thus, the AE boundary is a data manipulation boundary, as will be described in greater detail below, the boundaries can be viewed in different manners and otherwise can be used in different manners).

In some exemplary embodiments, the recommended AE boundary is set/identified in an arbitrary manner and not specific to a given recipient's preferences or needs. With reference to FIG. 3, the 70 dB level was set arbitrarily. A level of 60, 65, 70, 75, 80, 85, 90, etc., or any value in between or larger or lower can be used. Accordingly, in an exemplary embodiment, the AE boundary can be set utilizing statistical data, where, for example, based on a given demographic, etc., the level identified is a level where X percent of a population will have utilitarian results if such a level is utilized as the AE boundary.

In an exemplary embodiment, the remaining frequencies presenting a hearing loss are mapped to electrical stimulation, as with a typical cochlear implant irrespective of whether the recipient has residual hearing. In at least some exemplary embodiments, there is no overlap between the electric hearing in the acoustic hearing range. Of course, it is to be understood that in reality, the recipient can hear at, for example, 1000 Hz. The recipient simply cannot hear at a level above the threshold that was established (70 Hz). Accordingly, the thresholds and the boundaries detailed herein are fitting boundaries/hearing ability analysis boundaries. Another way of looking at this is that the thresholds in the boundaries detailed herein are boundaries meeting certain criteria, as is consistent with other types of data analysis/qualifications/quantifications (e.g., legally blind vs/totally blind, legally deaf vs. totally deaf, classifying someone as a genius or a moron based on IQ, classifying someone as a male or female based on chromosomes, classifying someone as an adult based on age (one can classify an adult based on law, based on reproductive ability, based on marriage status, etc.) etc.). It is noted that the aforementioned thresholds and/or boundaries are still real thresholds and/or boundaries. Such can be present in a prescription that is prepared for the hearing prosthesis or any other prostheses. By way of example only and not by way of limitation, there will be documentary evidence, in at least some scenarios of the teachings detailed herein, of the threshold and the boundary for a given recipient.

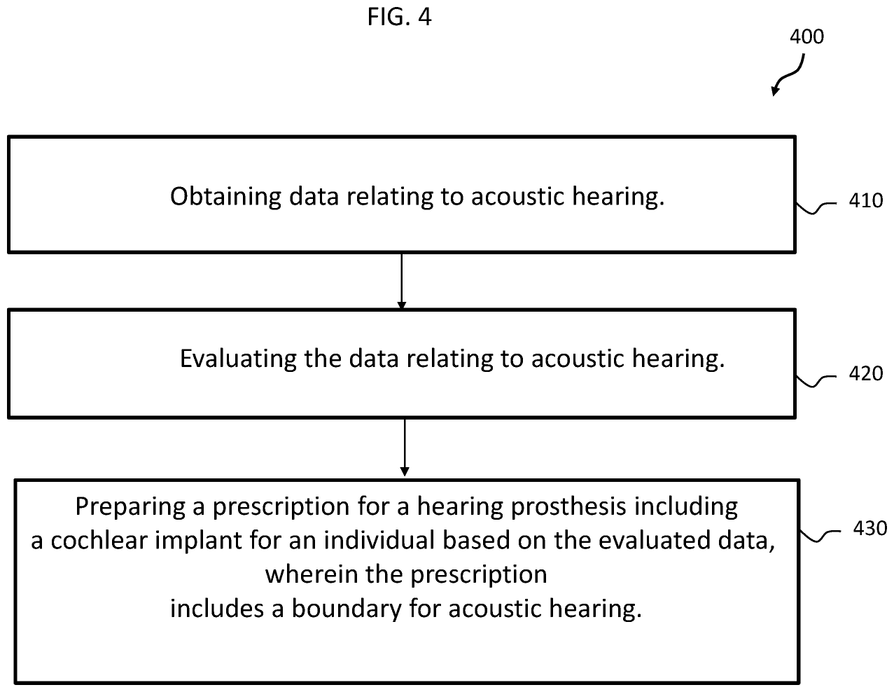

In view of the above, it can be seen that there can be exemplary methods, such as the method 400 represented by the flowchart in FIG. 4, which includes method action 410, which includes obtaining data relating to acoustic hearing. In an exemplary embodiment, method action 410 is executed by actually providing the recipient a hearing test in developing and audiogram. In an alternative embodiment, method action 410 is executed by obtaining the audiogram resulting from a previous test given to the recipient. Method 400 includes method action 420, which includes evaluating the data relating to the acoustic hearing, and method 430, which includes preparing a prescription for a hearing prosthesis including a cochlear implant for an individual based on the evaluated data, wherein the prescription includes a boundary for acoustic hearing.

With respect to the method 400, the boundary for acoustic hearing is established based on statistical data. In an exemplary implementation of method 400, the boundary for acoustic hearing is established based only on statistical data for a given population which is pertinent to the given recipient the subject of the method. In an exemplary implementation of method 400, there is no overlap between the electric hearing in the acoustic hearing, as the phrase “overlap” as utilized herein. In some implementations of method 400, there is an overlap, but the overlap is based entirely on statistical data for a given population which is pertinent to the given recipient the subject of the method.

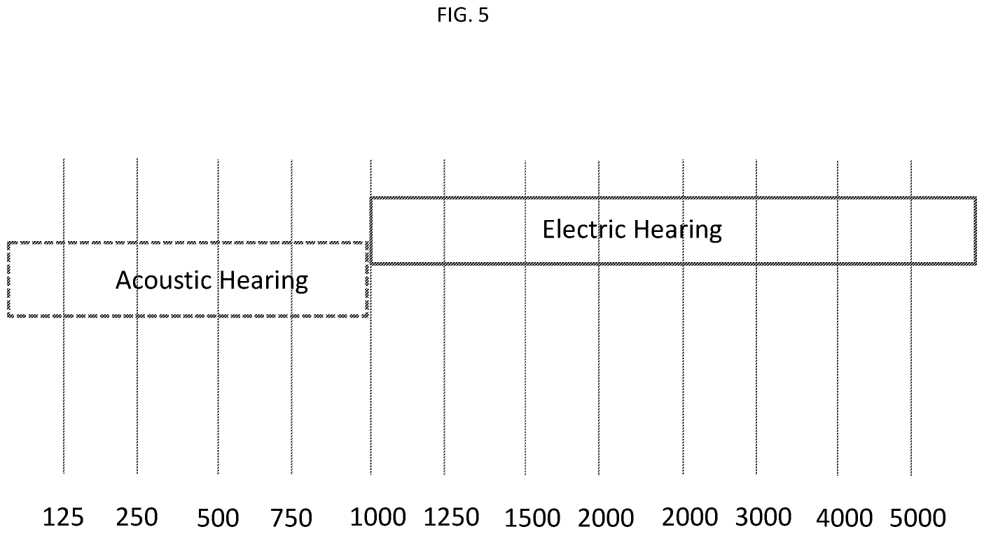

FIG. 5 shows an exemplary setting for a cochlear implant, an AE boundary and AE overlap resulting from method 400 based on the audiogram of FIG. 3. The diagram in FIG. 5 shows that electric hearing extends from and is inclusive of the 1000 Hz location. Thus, the electrode that is tonotopically mapped to frequencies of 1000 Hz would be a stimulating electrode. By way of example only and not by way of limitation, in an exemplary embodiment, this could be electrode 15 of a 22 electrode array, where electrodes 14-1 are lower frequency electrodes. Thus, the cochlear implant would be fitted such that electrodes 15, 16, 17, 18, 19, 20, 21, and 22, are identified as stimulating electrodes when the respective frequencies are experienced in the ambient environment, and electrodes 14 to 1 are identified as non-stimulating electrodes, where the electrodes are not stimulated when the respective frequencies are experienced in the ambient environment (e.g., a frequency below 1000 Hz). Note also that in an exemplary embodiment, where a so called short electrode is utilized, the electrode may only have electrodes 15-22 (or, more accurately, may only have electrodes for channels 15 to 22—such would be an eight electrode array). That said, in some scenarios where the short electrodes are standardized, where, for example, the number of electrodes are predetermined (e.g., 9 electrodes, 10 electrodes, etc.), only some of the electrodes will be stimulating electrodes (e.g., in a nine electrode array, only eight electrodes would be stimulating electrodes in the scenario).

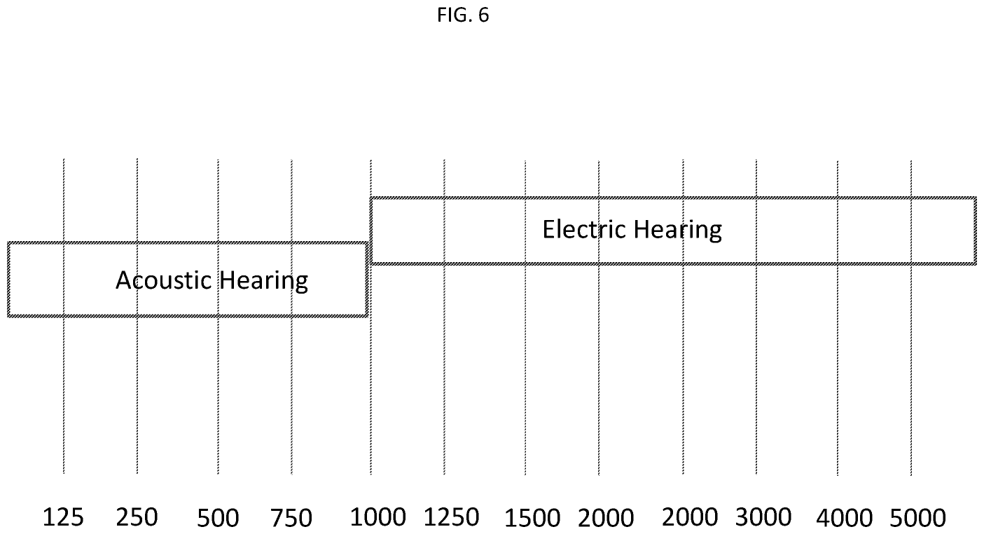

FIG. 5 shows that the box for acoustic hearing does not extend all the way to the 1000 Hz value. This is done simply to represent the fact that the electrical hearing is extended to an inclusive of the 1000 Hz. In reality, there is acoustic hearing at frequencies above 1000 Hz. Here, it is just that in this scenario, the AE boundary was established at frequencies where the audiogram dipped below 70 dB. Also, note that the acoustic hearing box is represented by dashes. This is because in method 400, the prostheses that was fitted was a single mode device in the form of a cochlear implant. Accordingly, the acoustic hearing range is not sat per se. Conversely, FIG. 6 shows a diagram for a multimode device according to FIG. 1A, where the acoustic stimulation device (e.g., ITE device) is also set as a result of the audiogram. Here, the acoustic hearing box is presented in a solid line to represent the fact that this is a setting, just as is the case with the cochlear implant portion of the multimode device.

Conversely, in some embodiments, there is a method that utilizes the subjective features of the recipient to develop the settings for the implant/to prepare a prescription for the hearing prosthesis. In an exemplary embodiment, there are methods, devices, and systems that enable, for example, clinicians in the clinic or remotely and/or by recipients themselves at home to customize their own AE boundaries and/or AE overlaps based on their own preferences and/or needs. In an exemplary embodiment, as opposed to a no comparison approach detailed above, there is a paired comparison approach. In an exemplary embodiment, the method(s) include competitions, in which, one option (EA boundary and EA overlap regime) is subjectively preferred over another option. The designs in question are programs that can be uniquely defined with different AE boundaries and AE overlaps.

An exemplary embodiment includes constructing unique settings using, for example, the following AE boundaries and AE overlaps:

TABLE I

| | |
|---|---|
| AE boundaries | audiogram drops below 60 dB HL, 65 dB HL, |
| | 70 dB HL, 75 dB HL, 80 dB HL, or 85 dB HL. |
| AE overlap | 0, 1, 2, 3, 4, or 5 stimulating electrodes |
| | within acoustic areas. |

Alternatively, the following boundaries and overlaps can be used:

TABLE II

| | |
|---|---|
| AE boundaries | audiogram drops below 57 dB HL, 60 dB HL, |
| | 63 dB HL, 68 dB HL, 70 dB HL, 75 dB HL, or |
| | 80 dB HL. |
| AE overlap | 0, 1, 2, 3, 4, 5, 6, 7 or 8 stimulating electrodes |
| | within acoustic areas. |

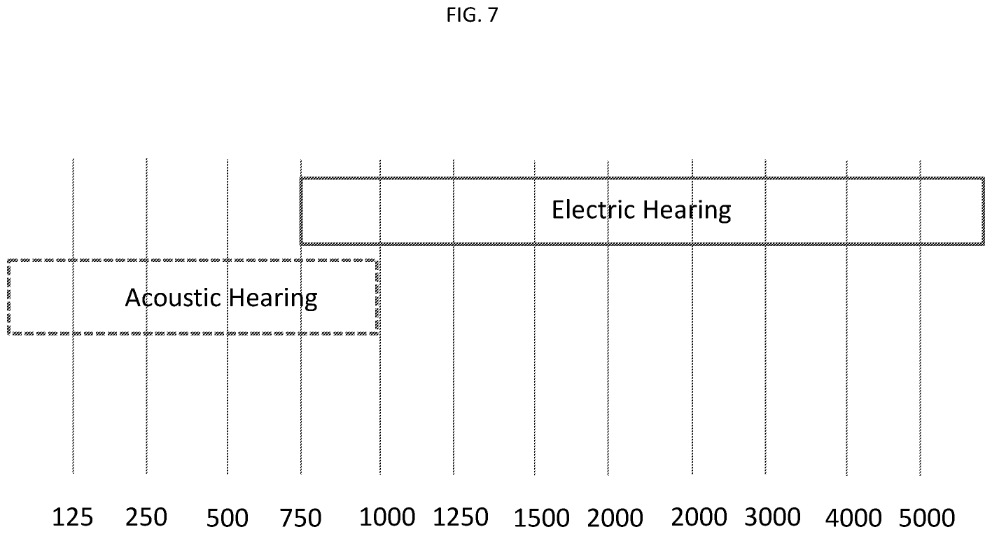

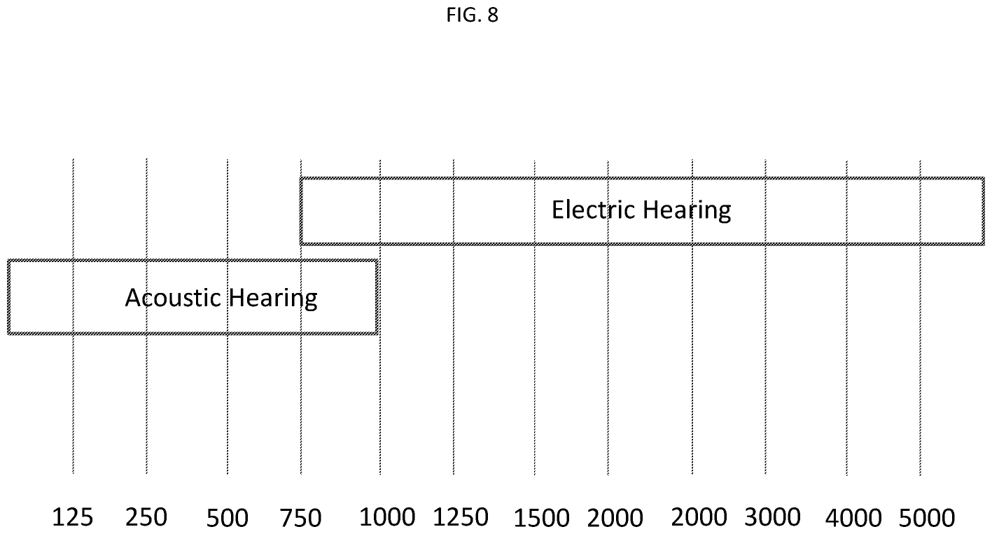

Briefly, with an AE boundary set at 70 dB and an AE overlap set at 0, such would correspond to FIG. 5, whereas with an AE overlap set at 2 electrodes (e.g., electrodes 13 and 14 in the hypothetical cochlear implant detailed above are also stimulating electrodes), such could correspond to that seen in FIG. 7, where, for example, electrode 14 and 13 are also stimulating electrodes, and such would stimulate at, for example, 825 Hz and 750 Hz. FIG. 8 depicts a diagram for a multi-modal device, where the ITE acoustic hearing aid is set to stimulate at frequencies from 1000 Hz to below (although there might be a cutoff as well at the lower end owing to general unpleasantness of hearing very low frequency sounds—note that the lower boundaries for the acoustic hearing in the upper boundaries for the electric hearing are not accurately depicted in the figures—in reality, the electric hearing would go potentially well above 5000 Hz, and the acoustic hearing could very well go below 60 Hz for example).

Consistent with the embodiments detailed above where subjective information is utilized to determine an overlap of the electric hearing with the acoustic hearing, a trial and error approach can be implemented where a given AE threshold and a given AE overlap is utilized to provide stimulus to the recipient utilizing the prostheses, and the recipient can indicate whether or not such is better, worse, or in different relative to another stimulus for another AE threshold and/or AE overlap. By way of example only and not by way of limitation, unique programs can be created from the parameters in table I above that will result in (N_{boundary})×(Nₒᵥₑᵣₗₐₚ)=(6) (6)=36 different paired comparisons. In an exemplary scenario, a full paired comparison trial will require that every option/program be compared against every other program (in this case, 36 comparisons). The resulting matrix of comparisons will be large and time-consuming within the context of a clinical visit or even an at home task (e.g., 1260 scenarios). Moreover, an exemplary comparison regime can include doubling the comparisons to randomize the program order (e.g., program A vs. program B and program B vs. program A), thus increasing the number of possible permutations. The matrix of comparisons is subject to change depending on a patient's sensitivity to different parameter values.

Note that with respect to the AE boundary, in some instances, the AE boundary can be considered to begin at the last electrode that is stimulated in a fitting/analysis regime where it is desired that there be no overlap. In this regard, all frequencies below that last electrode stimulated would be acoustic hearing. Again, this is a standards-based regime. Because the value of the threshold for acoustic hearing and electric hearing could be different depending on the personal preferences of the audiologist or the beliefs of the organization for which the audiologist works or the like, or the manufacture of the prostheses, what would be a threshold for one audiologist working for one company or utilizing one corporation's product (prostheses product) could be different for another, even for the same recipient. Accordingly, by defining the threshold/AE boundary as the location where the electrodes begin to be activated for a regime where there is no overlap, the definition of overlap and the definition of AE boundary become standardized. In this regard, one can consider the AE boundary to be a cochlear implant fitting boundary where there will always be an electrode that will stimulate/the boundary at which the pairwise comparison will not consider “deactivating” an electrode.

An exemplary embodiment includes utilizing techniques that can be used in studies of preferences, attitudes, voting systems, and judgment to cull or otherwise reduce the matrix of comparisons to a more manageable and less exhausting level. Using such can result in the ability to not have to “settle” for a zero overlap fitting and/or a minimal overlap fitting and/or a statistically based (only) fitting, and/or not have to settle for a paired down trial and error comparison approach that is based on guesstimation/educated guess, or instinct of the person performing the analysis (e.g., clinician).

More particularly, acoustic stimulation at frequencies where the recipient can generally hear at a utilitarian and/or non-legally deaf level can have utilitarian value. That is, an overlap of electrical stimulation with the acoustic hearing range can be desirable in some instances. However, there are limits on the amount of overlap, owing to the possibility that the electric hearing can confuse or otherwise detract from the acoustic hearing. The teachings detailed herein can provide methods, devices, and systems that can provide an accelerated process to identify a utilitarian AE threshold in combination with the utilitarian AE overlap relative to that which would be the case with respect to the brute force method of trying each possible combination. A full paired comparison (brute force) strategy requires a subject to listen to every design versus every other design and judge between the two. This is untenable and results in, (36×35) 1260 comparisons. In an exemplary embodiment, the mechanics of a tabu searcher utilized, where the number of comparisons can be pared down considerably relative to that which might otherwise be the case utilizing the brute force method. By way of example only and not by way of limitation, relative to the brute force method (without the randomization of the order), the number of combinations that are applied are 50, 51, 52, 53, 54, 55, 56, 67, 68, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9 or more percent lower or any value or range of value therebetween in 0.01 increment (e.g., 66.67 to 93.33 percent, 99.89 percent, etc.). In an exemplary embodiment, a double elimination, a triple elimination, a 4, 5, 6, 7, 8, 9, 10, 11, 12, 13, 14, or 15 or more elimination rule can be imposed such that when a given program of AE overlap and AE threshold loses Y (the elimination value) pairwise comparisons, it is placed on the tabu list and removed from the total number of programs. Therefore, the comparison matrix diminishes according. In terms of implementation, rather than assigning the AE boundary and the AE overlap, the fitting would offer a paired comparison tasks to optimize these parameters.

For example, where Y is 2 (a double elimination rule), using the parameter combinations associated with Table I above, for a recipient to navigate through the different designs in this population in search of an optimal setting (design), utilizing a paired (or pairwise) comparison approach allows listeners to hear two designs head-to-head, thus the paired comparison. Using a tabu search, the number of comparisons can be reduced. Utilizing a tabu strategy can maintain the running status account of all designs and how they fared in paired comparison trials. Because Y=2, there is a double elimination approach, in which designs are considered tabu if they have been rejected twice. Designs that are considered tabu are then removed from the full population of designs and not used in subsequent paired comparison trials.

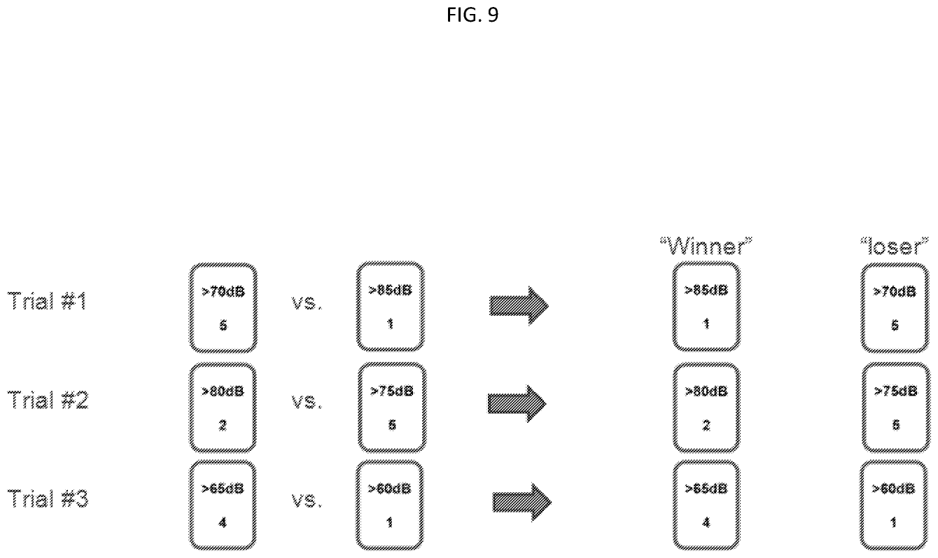

An exemplary run of the paired comparison/tabu approach is as follows. Sound samples are processed by two designs selected randomly from the design population and then presented to the listener. The listener subsequently selects the “winner” of the trial. In the background, the software (or human or any device, system, and/or method that can enable such) is maintaining status information regarding each design. This is represented in the schematic presented in FIG. 9, where the AE overlap is set at zero.

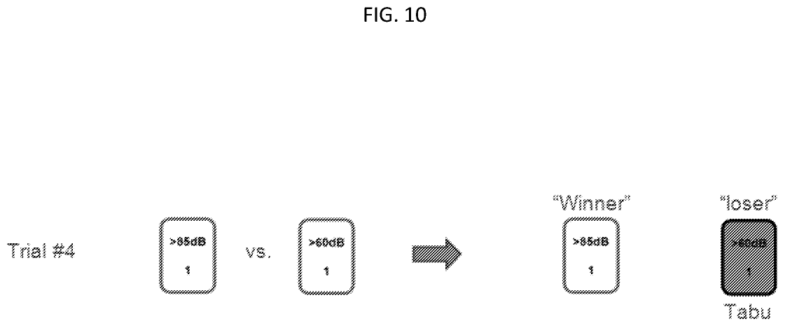

Up to this point, no randomly selected designs have been duplicates. In the next exemplary trial, a duplicate design is shown to lose for a second time, as represented in FIG. 10. Having lost twice, this design is considered tabu and removed from the design population. As a result, the design population shrinks slightly from 36 to 35 (no designs that include 60 dB at zero overlap, but 60 dB will be used for 1, 2, 3, 4, 5 overlap) and the number of potential comparisons a recipient must make shrinks from 1260 to 1190 (excluding the comparisons that have already been made). As the trials proceed, the design population and “necessary” comparisons will continue to reduce.

Table III below depicts some exemplary elimination scenarios.

TABLE III

| AE | AE | AE | | |
|---|---|---|---|---|
| Overlap | Threshold | Threshold | Winner | Eliminated |
| 0 | 70 | 85 | 85 | |
| 0 | 80 | 75 | 80 | |
| 0 | 65 | 60 | 65 | |
| 0 | 85 | 60 | 85 | 60 |
| 0 | 70 | 75 | 75 | 70 |
| 1 | 65 | 85 | 85 | |
| 1 | 75 | 80 | 80 | |
| 1 | 65 | 60 | 60 | 65 |
| 1 | 80 | 60 | 80 | |
| 1 | 70 | 75 | 70 | 75 |

Note that simply because 60 Hz was eliminated with the zero overlap did not eliminate such for the 1 overlap. This is also the case with the 70 Hz at zero overlap.

Note also that the tabu algorithm can be used for electrode overlap elimination. Table IV below shows an exemplary scenario of such use:

TABLE IV

| AE | AE | AE | | |
|---|---|---|---|---|
| Overlap | Overlap | Threshold | Winner | Eliminated |
| 0 | 1 | 70 | 1 | |
| 0 | 2 | 70 | 0 | |
| 0 | 3 | 70 | 0 | |
| 0 | 4 | 0 | 4 | 0 |
| 1 | 2 | 70 | 1 | |
| 1 | 3 | 70 | 3 | 1 |

An embodiment includes expediting the comparison process such that the full paired comparison process is expedited by exploiting parameter monotonicity. By way of example only and not by way of limitation, if a determination can be made that that no E/A Boundary greater than 75 dB (80 dB and 85 dB spec to Table I) have never been preferred in a paired comparison, some exemplary embodiments can conclude that the natural ceiling for this parameter in terms of this listener is 75 dB and all designs comprising an AE Boundary values of 80 dB and 85 dB may be removed from the design population. In some embodiments, this conclusion is not made as a matter of routine, and is only made following a statistically appropriate number of comparisons (e.g., implemented only after a threshold number of comparisons).

In view of the above, it can be seen that there are devices, systems, and methods of determining electro/acoustic parameters (e.g., AE boundary, AE overlap) tailored to the needs/preferences of the recipient, as oppose to those using statistical based data/or using subjective testing without considering overlap or at least only considering minimal overlap). Such can functionally optimize the parameters of AE boundary and Ae overlap subjectively for a given recipient. This thus differentiates from such things as Custom Sound™, which automatically creates a MAP with a “minimal overlap” and a frequency boundary where hearing loss is determined to be 70 dB HL and frequencies with a hearing loss greater than 70 dB HL, are mapped to areas of electrical stimulation, and frequencies at which hearing loss is less than 70 dB HL are mapped to areas of acoustic stimulation. The embodiments detailed herein using the advanced comparison techniques to not automatically create a MAP with a “minimal overlap” and a frequency boundary where hearing loss is determined to be 70 dB HL, and frequencies with a hearing loss greater than 70 dB HL are mapped to areas of electrical stimulation and frequencies at which hearing loss is less than 70 dB HL are mapped to areas of acoustic stimulation. Instead, in some embodiments, each recipient optimizes their own frequency boundary and overlap characteristics based on a subjective and functional manipulation based on their hearing feedback. Tailoring the AE parameters for each recipient can improve perceived sound quality and performance relative to the fixed approach detailed above based on statistical data/limited overlap.

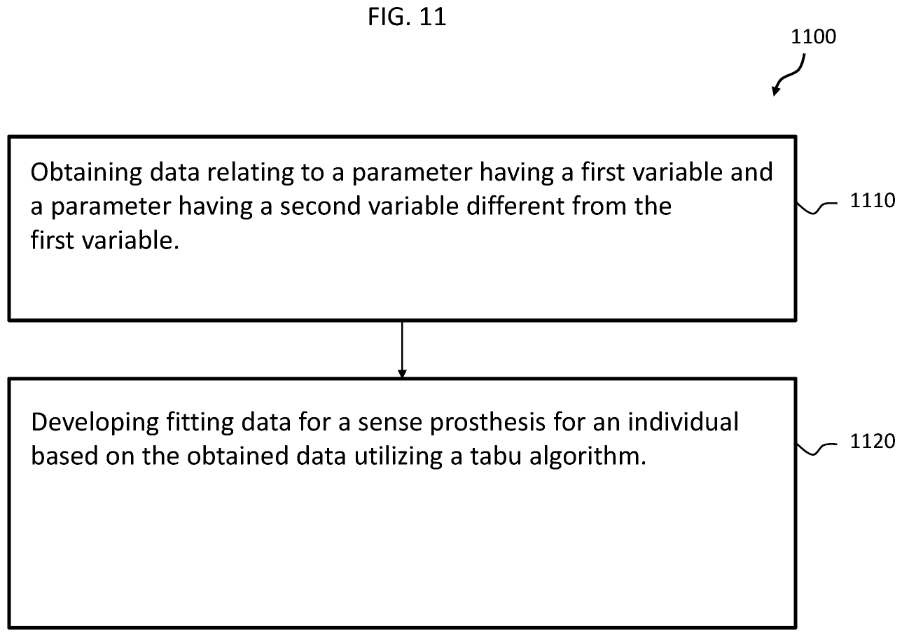

In view of the above, it can be seen that an embodiment can include a method 1100, represented by the flowchart of FIG. 11, which can include method action 1110, which includes obtaining data relating to a parameter having a first variable and a parameter having a second variable different from the first variable. In an exemplary embodiment, the parameter having a first variable is the AE threshold parameter, which can be various values as detailed above, and the parameter having a second variable is the AE overlap parameter, which also can be various values as detailed above. The action of obtaining the data in method action 1110 can correspond to actually giving a recipient a hearing test or the like while using the hearing prostheses, and then receiving input from the recipient regarding the preferences of a given comparison. That said, the action of obtaining the data in method action 1110 can correspond to simply receiving data indicative of input from the recipient regarding the preferences of a given comparison. Method 1100 also includes method action 1120, which includes developing fitting data for a sense prosthesis (again, the teachings detailed herein are not limited to only hearing prostheses, but can also include retinal prostheses, etc.) for an individual based on the obtained data utilizing a tabu algorithm. While it is to be noted that in the embodiments described above, the taboo algorithm is utilized so as to establish what components of the comparisons will and/or will not be presented. Still, because the obtained data is manipulated utilizing a tabu algorithm, the fitting data for the sense prostheses is still based on the utilization of a tabu algorithm. That said, in an exemplary embodiment, where, for example, the sense prosthesis includes a cochlear implant (which means that the sense prosthesis can be a multimode prosthesis or can be a single mode prosthesis), and the obtained data is data relating to electric hearing and data relating to acoustic hearing, the action of obtaining data relating to electric hearing and data relating to acoustic hearing is controlled at least in part based on the utilize taboo algorithm. In this regard, this is because, in some embodiments, as noted above, the tabu algorithm is utilized to determine what is and is not to be presented to the recipient in the comparisons. Thus, the obtained data will be based on the utilized tabu algorithm.

While the above exemplary embodiment has focused on a multimodal prostheses, as noted above, the teachings detailed herein can be utilized for any type of sense prostheses, including single mode hearing prostheses. The tabu algorithm, etc., can be utilized for any subjective design approach in which the recipient of the prostheses compares two options, such as a pairwise comparison, and determines the option deemed to be more utilitarian or otherwise preferred. Again, as noted above, this can be done with respect to a retinal prostheses. Also, consistent with the teachings detailed above, a taboo algorithm could be utilized to compare whether it is better to utilize all electrodes of an electrode array (e.g., all 22 electrodes of a 22 electrode array), or at least most of them (e.g., 21 or 20 or 19—there are often a few channels that do not work for whatever reason), verses utilizing subsets of electrodes of the electrode array (e.g., 10 electrodes of the 22 electrodes, 8 of the 22, 12 of the 22, etc.). Accordingly, in an exemplary embodiment, the tabu algorithm could have a first variable constituting a first number of electrodes of the electrode array and a second variable constituting a second number of electrodes of the electrode array and/or a different set of electrodes (same number, but different electrodes), and so on. Alternatively and/or in addition to this, another variable can be the charge applied to the electrodes and/or to individual electrodes, while another variable can be the number of maxima, while another variable can be stimulation rate, etc. accordingly, as can be seen, for a cochlear implant, the number of variables can be quite large. Accordingly, the teachings detailed herein can be utilized to fit the cochlear implant utilizing a tabu algorithm instead of a brute force method. In an exemplary embodiment, there can be one, two, three, four, five, six, seven, eight, nine and/or ten sub variables for any one or more of the aforementioned variables just detailed with the cochlear implant.

To be clear, in at least some exemplary embodiments, any subjective parameter that can be varuied or is a variable that otherwise would be optimized utilizing a brute force method can instead be optimize utilizing the tabu algorithms detailed herein for any type of prostheses.

Consistent with the teachings above, the action of developing fitting data can include or otherwise correspond to preparing a prescription for the hearing prosthesis for the individual. Again, the prepared prescription can be contemporaneous with the application of the fitting settings, or the prescription can be generated and then almost immediately applied to the implant. For example, in an exemplary embodiment where the methods detailed herein are executed where the audiologist or the server or the computer that performs some of the method actions is located remotely from the recipient, upon the completion of the method actions that identify the optimal AE boundary and the optimal AE overlap, a prescription can be generated identifying such (the prescription can include other things, such as threshold levels and comfort levels—the teachings detailed herein can be combined with other types of fitting methods), and the “prescription” can be transmitted via the Internet or the like to the recipient and/or to the prosthesis. The recipient can then set the prosthesis based on a prescription and/or the recipient can have a device that will do so itself (e.g., a smart phone or a personal computer that is in communication with the prostheses, the prostheses itself, etc.). This could happen within seconds of the transmission of the prescription to the recipient.

Consistent with the teachings detailed above, in at least some exemplary embodiments, the obtained data obtained in method action 1110 respectively includes at least two hybrid parameters. Also consistent with the above, in an exemplary embodiment, the method begins with a first number of possible combinations of the first parameter with the second parameter (e.g., 1260 detailed above with respect to Table I), and the use of the tabu algorithm results in the obtained data relating to electric hearing and the obtained data relating to acoustic hearing having a second number of combinations of the first parameter with the second parameter that is less than 80% of the first number. Again, in some embodiments, the use of the tabu algorithm (or whatever algorithm is used) results in the obtained data relating to electric hearing and the obtained data relating to acoustic hearing having a second number of combinations of the first parameter with the second parameter that is less than 50, 51, 52, 53, 54, 55, 56, 67, 68, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9 or more percent lower or any value or range of value therebetween in 0.01 increment (e.g., 66.67 to 93.33 percent, 99.89 percent, etc.) of the first number.

In some embodiments, method action 1110 includes applying paired comparison tasks and receiving input based on the tasks, wherein respective comparison tasks have at least one variable that is different with respect to the first variable and/or the second variable. In some embodiments, both variables are different. It is noted that while this embodiment is presented with respect to a two-dimensional variable string, a 3, 4, 5, or more variable string can be paired, and one, 2, 3, 4 or 5 or more can be different in each comparison. Some additional details of this are described below.

Also, consistent with the above, it can be seen that in an exemplary embodiment, method 1100 provides optimization of a frequency boundary for electro-acoustic hearing and an overlap of electric hearing based on subjective feedback from the recipient.

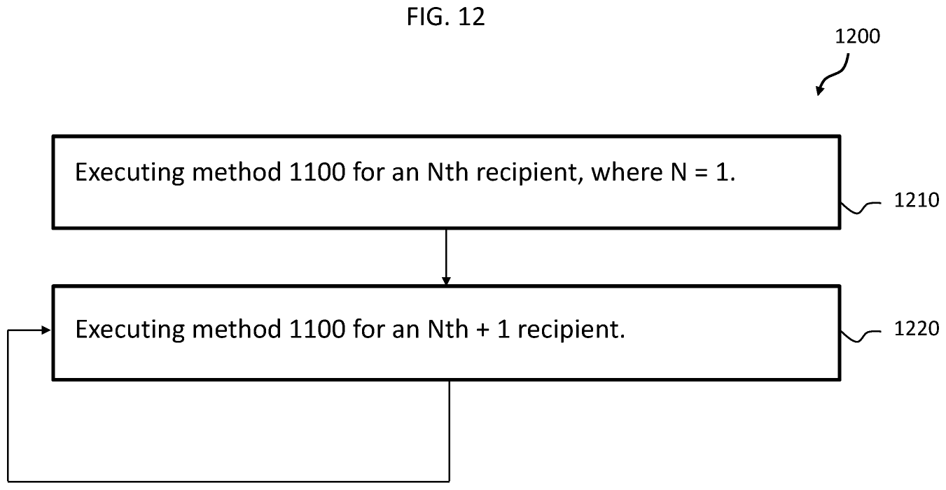

FIG. 12 presents an exemplary algorithm for an exemplary method, method 1200, which includes method action 1210, which includes executing method 1100 for an Nth recipient, where N=1. The method goes on to execute method action 1220, which includes executing method 1100 for another recipient (Nth+1). Method 1200 goes on to reexecute method action 1220 for another recipient (now N=3), and so on, until N equals a given value (e.g., 10, 11, 12, 13, 14, 15, 16, 17, 18, 10, 20, 25, 30, 35, 40, 45, 50, 55, 60, 65, 70), and this can be executed in a timeframe lasting starting from N=1 to the last N of 1, 2, 3, 4, 5, 6, 7, 8, 9, 10, 11, 12, 13, 14, 15, 16, 17, 18, 19, 20, 25, 30, 35, 40, 45, 50, 55, 60 weeks, 14, 15, 16, 17, 18, 19, 20, 25, 30, 35, 40, 45, 50, 55, 60, 65, 70, 75, 80, 85 or 90 months, or more or less. In an exemplary embodiment, less than 1, 2, 3, 4, 5, 6, 7, 8, 9, 10, 11, 12, 13, 14, 15, 16, 17, 18, 19, 20, 25, 30, 35 or 40 percent of recipients will have the same AE boundary and AE threshold. Indeed, in some embodiments, none of the recipients will have the same AE boundary and AE threshold.

In an exemplary embodiment, there is an exemplary method that comprises setting a cochlear implant to operate based on data based on a first number of comparisons between respective data sets respectively including combined data for electric stimulation to evoke a hearing percept and data for acoustic stimulation to evoke a hearing percept, wherein the first number of comparisons is less than a total number of possible comparisons resulting from all possible permutations of the controlled variables that make up the respective data sets. In this regard, with respect to the scenarios of Table I above, the total number of possible comparisons amounts to 1260 (without the doubling of the comparisons—if doubling, then this number would be higher, as would also the first number of comparisons—the two are thus linked as to the implementation method of the comparison). As demonstrated above, the first number of comparisons will be significantly less than the total number (e.g., 90% less, more than 90% less). Again, in an exemplary embodiment, the first number of comparisons is developed as a result of a tabu search. As detailed above, it is the tabu search that results in the data sets that will be provided to the recipient (and thus will not be provided to the recipient), and the reduction in the data sets that will be provided to the recipient will result in the number of the comparisons being lower than the total number of possible comparisons.

Consistent with the embodiment described above with respect to fitting a cochlear implant, the combined data relating to electric stimulation and data relating to acoustic stimulation includes a first parameter that includes electro-acoustic boundary data and a second parameter that includes an electro-acoustic overlap data and the controlled variables are an electrical-acoustic boundary threshold and an electrical-acoustic overlap range and the electrical-acoustic overlap range is a number of electrodes within the acoustic range (wherein the acoustic range is, in some embodiments, the range where an audiogram indicates the recipient can hear frequencies below a certain threshold). Also consistent with the teachings above, the method of fitting the cochlear implant includes evoking respective hearing percepts using the cochlear implant to develop the data of the respective data sets and the action of evoking respective hearing percepts and the action of setting the cochlear implant is executed by a recipient of the hearing prosthesis. In this regard, it is noted that in at least some exemplary embodiments, the techniques detailed herein can be executed by the recipient autonomously without the need of a clinician or the like. By way of example only and not by way of limitation, the methods detailed herein can be executed by a recipient utilizing a personal computer or a portable computing device, such as a smart phone or the like, with an application program thereon that can enable the methods detailed herein executed, at least in part, by the computer. Moreover, in at least some exemplary embodiments, the prosthesis itself can be programmed to implement some or more or all of the methods detailed herein. In an exemplary embodiment, prosthesis 200 can be configured to provide a recipient with various combinations of stimuli, receive input from the recipient indicating preferences, develop new combinations/future combinations based on the received input, and then fit the prostheses based on the received input. Any device, system, and/or method that can implement the teachings detailed herein can be utilized in at least some exemplary embodiments.

Accordingly, consistent with the teachings above, in at least some exemplary embodiments, there is a non-transitory computer readable medium having recorded thereon, a computer program for executing a method, the program including code for executing a tabu algorithm to develop respective test data to fit a sense prosthesis to a recipient, wherein the respective test data includes respective sensory percepts to be evoked using the prosthesis, wherein the respective test data is at least two dimensional data. By way of example only and not by way of limitation, the two dimensional data can correspond to AE threshold and AE overlap. That said, in some embodiments, the respective test data is at least three dimensional test data. In an exemplary embodiment, the test data can include the aforementioned AE threshold and AE overlap, but can also include, for example, different current levels for the electrical stimulation for the electrodes. Moreover, in an exemplary embodiment, the test data can include the aforementioned AE threshold and AE overlap, but can also include, for example, different volumes for the ITE device for the acoustic stimulation, at least for embodiments that utilize a multimodal device. A four dimensional data regime can correspond to, for example, the AE threshold, the AE overlap, different current levels, and different volumes for the ITE device,

In an exemplary embodiment, the medium avoids the application of brute force test data to fit the sense prosthesis. That is, in an exemplary embodiment, the prosthesis could be fitted utilizing a force method, and the medium fits the hearing prosthesis without doing so, thus providing efficiency relative to the brute force method.

Of course, consistent with the exemplary embodiment of FIG. 1A, the sense prosthesis is a cochlear implant, and the action of fitting the sense prosthesis corresponds to customizing an electro-acoustic boundary and an electro-acoustic overlap of the hearing prosthesis. Accordingly, in some embodiments, the respective test data includes a first parameter that includes electro-acoustic boundary data and a second parameter that includes an electro-acoustic overlap data.

That said, in some alternate embodiments, where, for example, the sense prosthesis is a retinal implant, the action of fitting the sense prosthesis corresponds to customizing an electro-optic boundary and an electro-optic overlap of the retinal prosthesis.

In some embodiments of the medium described above, the tabu algorithm applies a double elimination rule for at least a portion of its use. By way of example only and not by way of limitation, a double elimination rule can be applied for the first 10 eliminations, and then another elimination rule, such as a triple, quintuple, etc., elimination rule, can be applied for other eliminations, etc. That said, in some embodiments, a double elimination rule is applied all the time. In some embodiments, a triple elimination rule is applied all the time. In some embodiments, a tabu algorithm is utilized until there are a certain number of remaining comparison scenarios, and then a force method is implemented for the remaining scenarios.

In some embodiments, the medium has code for automatically initiating a sensory percept by the sense prosthesis based on the obtained test respective data and code for evaluating subject input from the recipient based on the initiated sensory percept, wherein the code for executing the tabu algorithm uses the evaluated subjective input. Such can have utilitarian value with respect to an automated or semiautomated fitting procedure/device, such as the programming system 260 detailed above, or a smart phone or the like programmed to execute some or more or all of the teachings detailed herein, or a prostheses that is configured to execute such.

Thus, in some embodiments there is a fitting system, such as system 260 above, or a smartphone or a prosthesis, or even a remote server based system, comprising a first sub-system configured to cause respective stimulus groups to be applied by a sensory prosthesis (e.g., a hearing prosthesis, a retinal prosthesis, etc.) to a recipient of the sensory prosthesis. In an exemplary embodiment, the respective stimulus groups are groups of AE threshold plus AE overlap stimulations. In an exemplary embodiment, the first sub-system is a system that communicates with the hearing prostheses via, for example, line 284, and provides a signal thereto that causes the prostheses to operate to evoke a hearing percept based on the respective stimulus group. It is noted that in some embodiments, the fitting system communicates with the prostheses wirelessly. Still further, in some embodiments where the fitting system is integrated into the prostheses, the fitting system is wired to the prostheses, at least in some embodiments.

Still further, in an exemplary embodiment of this fitting system, there is a second sub-system configured to automatically determine the respective stimulus groups to be applied from a pool of potential respective stimulus groups that is larger than what the first sub-system will cause to be applied. Again, as noted above, with respect to Table I, there are 36 stimulus groups. This fitting system will reduce those stimulus groups that are applied. In at least some exemplary embodiments, the second sub-system utilizes a tabu algorithm to determine the respective stimulus groups.

In at least some exemplary embodiments, the pool of potential respective stimulus groups includes at least 9 possible stimulus respectively having at least two variables (e.g., EA boundary/threshold, EA overlap. That said, the pool can include 10, 11, 12, 13, 14, 15, 16, 18, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 or more groups, the respective groups having 2, 3, 4, 5, 6, 7, 8, 9 or 10 variables or more, and one or more or all of the respective variables having at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 or more possibilities (based on standards—an EA boundary of 77.7 dB and 77.8 dB as a variable is unlikely, as the ear likely cannot distinguish one better than the other)—note that one group can have a different number of variables than another, and one or more of the variables can have a different number of possibilities than the other)). The variables are fitting variables in at least some embodiments.

In some exemplary embodiments, the second sub-system is configured to provide respective stimulus groups to the recipient in an abbreviated paired comparison trial. This as opposed to a full paired comparison trial (brute force).

In an exemplary embodiment, the fitting system further comprises a third sub-system configured to receive respective data based on perceptions of the recipient resulting from the respective stimulus groups (e.g., which of the pairs is better than the other or worse than the other). In an exemplary embodiment, this can be a voting button used in paired comparisons. This can be a touch screen, or a microphone, or any device system and/or method that can enable the recipient to input data into the fitting system. In an exemplary embodiment, the second sub-system utilizes the received respective data based on the perceptions to automatically determine the respective stimulus groups to be applied such that the determined respective stimulus groups to be applied is lower than the number of potential respective stimuluses groups (where the values for lower have been detailed above for some embodiments).

In an exemplary embodiment, of the fitting system, the sense prosthesis includes a cochlear implant and the respective stimulus groups are respectively based on respective electro-acoustic boundaries and respective electro-acoustic overlap.

It is noted that in some embodiments, the subjective determination of the AE boundary for a given recipient can be based on data where there is also electric hearing applied for that data. In this regard, for example, when developing the groups that include an AE overlap, there will be stimulation of at least one electrode at frequency locations within the cochlea that are below the acoustic hearing threshold set for the test. This is as opposed to other types of fitting methods where the data relating to acoustic hearing may not have an electric hearing component. Put another way, when the recipient is asked to evaluate a given group compared to another group, it is likely that for the vast majority, and certainly for the most number of scenarios, the given group will include activation of the cochlear implant for a given frequency, even though that frequency is below the AE boundary. Indeed, with respect to the embodiments of Table I, five out of 6 groups will have the activated electrode.

It is noted that the teachings detailed herein can be applicable for other parametric or program fitting applications other than the AE boundary and AE overlap program. In some embodiments, a given recipient can customize their own fitting based on their own unique hearing needs/desires. For example, a pairwise comparison task can be used to optimize cochlear electrode deactivation. Also by way of example, a pairwise comparison can be used to optimize frequency electrode associations. The the tabu list mitigation approach detailed herein can be used in these instances. Embodiments extend to any scenario where, tabu list mitigation can expedite subjective analysis.

Is briefly noted that in some exemplary embodiments, presentation of groups to the recipient can be presented in a “intelligent” manner. For example, in the example detailed above where for trial 1 the loser was 70 dB, in an exemplary embodiment, the next trial could also include 70 dB in the pairwise comparison. This would have the effective immediately eliminating a group (potentially). That said, some other embodiments are presented in a more random manner.

It is noted that at least some exemplary embodiments are strictly limited to a paired comparison task. Some embodiments are strictly limited to paired comparisons of two different designs/programs for the prosthesis. This as opposed to, for example, providing three or four or five different groups or more to the recipient and having the recipient choose from amongst the groups. Indeed, some implementations of managing large groups in a subjective fitting setting or the like can utilize a genetic algorithm or the like. Some embodiments of the teachings detailed herein explicitly exclude the use of a genetic algorithm and/or a Bayesian algorithm. Genetic algorithms and Bayesian algorithms have their purpose, and certainly can have utilitarian value. However, the teachings detailed herein provide utility in a different manner and approach the problem of managing a large number of possible permutations utilizing a tabu algorithm.

Any disclosure of any method action detailed herein corresponds to a disclosure of a device and/or a system and/or computer code for executing that method action. Any disclosure of any method of making an apparatus detailed herein corresponds to a resulting apparatus made by that method. Any functionality of any apparatus or program product or computer code detailed herein corresponds to a method having a method action associated with that functionality. Any disclosure of any apparatus and/or system and/or computer code detailed herein corresponds to a method of utilizing that apparatus and/or system and/or code. Any feature of any embodiment detailed herein can be combined with any other feature of any other embodiment detailed herein providing that the art enables such, and it is not otherwise noted that such is not the case. Any one or more features disclosed herein can be explicitly excluded from use with any other one or more features disclosed herein.

While various embodiments of the present invention have been described above, it should be understood that they have been presented by way of example only, and not limitation. It will be apparent to persons skilled in the relevant art that various changes in form and detail can be made therein without departing from the scope of the invention.

1. A method, comprising:
obtaining data relating to a parameter having a first variable and a parameter having a second variable different from the first variable; and
developing fitting data for a sense prosthesis for an individual based on the obtained data utilizing a tabu algorithm.

2. The method of claim 1, wherein:
the sense prosthesis includes a cochlear implant; and
the obtained data is data relating to electric hearing and data relating to acoustic hearing.

3. The method of claim 2, wherein:
the action of obtaining data relating to electric hearing and data relating to acoustic hearing is controlled at least in part based on the utilized tabu algorithm.

4. The method of claim 1, wherein:
the obtained data includes at least two hybrid parameters.

5. The method of claim 3, wherein:
the method begins with a first number of possible combinations of the first variable with the second variable, and the use of the tabu algorithm results in the obtained data relating to electric hearing and the obtained data relating to acoustic hearing having a second number of combinations of the first variable with the second variable that is less than 80% of the first number.

6. The method of claim 1, wherein:
the action of obtaining data includes applying paired comparison tasks and receiving input based on the tasks, wherein respective comparison tasks have at least one variable that is different with respect to the first variable and/or the second variable.

7. The method of claim 1, wherein:
the action of developing fitting data includes preparing a prescription for the hearing prosthesis for the individual.

8-10. (canceled)

11. A method, comprising:
setting a cochlear implant to operate based on data based on a first number of comparisons between respective data sets respectively including combined data for electric stimulation to evoke a hearing percept and data for acoustic stimulation to evoke a hearing percept, wherein the first number of comparisons is less than a total number of possible comparisons resulting from all possible permutations of the controlled variables that make up the respective data sets.

12. The method of claim 11, wherein:
the first number of comparisons is developed as a result of a tabu search.

13. The method of claim 11, wherein:
the first number of comparisons is less than 70% of the total number of possible comparisons.

14. The method of claim 11, wherein:
the first number of comparisons is less than 95% of the total number of possible comparisons.

15. (canceled)

16. The method of claim 11, wherein:
the controlled variables are an electrical-acoustic boundary threshold and an electrical-acoustic overlap range.

17. (canceled)

18. The method of claim 11, further comprising:
evoking respective hearing percepts using the cochlear implant to develop the data of the respective data sets, wherein
the action of evoking respective hearing percepts and the action of setting the cochlear implant is executed by a recipient of the hearing prosthesis.

19-28. (canceled)

29. A fitting system, comprising:
a first sub-system configured to cause respective stimulus groups to be applied by a sensory prosthesis to a recipient of the sensory prosthesis; and
a second sub-system configured to automatically determine the respective stimulus groups to be applied from a pool of potential respective stimulus groups that is larger than what the first sub-system will cause to be applied.

30. The fitting system of claim 29, wherein:
the sensory prosthesis is a hearing prosthesis.

31. The fitting system of claim 29, wherein:
the sensory prosthesis is a retinal prosthesis.

32. The fitting system of claim 29, wherein:
the second sub-system utilizes a tabu algorithm to determine the respective stimulus groups.

33. The fitting system of claim 29, wherein:
the pool of potential respective stimulus groups includes at least 4 possible stimulus groups respectively having at least two variables.

34. The fitting system of claim 29, wherein:
the second sub-system is configured to provide respective stimulus groups to the recipient in an abbreviated paired comparison trial.

35. The fitting system of claim 29, further comprising:
a third sub-system configured to receive respective data based on perceptions of the recipient resulting from the respective stimulus groups, wherein
the second sub-system utilizes the received respective data based on the perceptions to automatically determine the respective stimulus groups to be applied such that the determined respective stimulus groups to be applied is at least 50% lower than the number of potential respective stimuluses groups.

36. (canceled)
